(19) United States
(12) Patent Application Publication
(10) Pub. No.: US 2018/0224673 A1
(43) Pub. Date: Aug. 9, 2018

(54) Relaxation and meditation eyewear

(71) Applicant: Robert Therrien, Trois-Rivieres (CA)

(72) Inventor: Robert Therrien, Trois-Rivieres (CA)

(21) Appl. No.: 15/749,503

(22) PCT Filed: Sep. 13, 2016
(86) PCT No.: PCT/CA2016/000231

§ 371 Date: Feb. 1, 2018

Related U.S. Application Data
(60) Provisional application No. 62/283,924, filed on Sep. 16, 2015.

(51) Int. Cl. G02C 7/14; A61M 21/02; G02C 11/02; G02C 11/04; G02C 11/00

(52) U.S. Cl. CPC G02C 7/14; A61M 21/02; A61M 2021/0044; G02C 11/04; G02C 11/10; G02C 11/02

(57) ABSTRACT

A relaxation and meditation eyewear has temples, opaque lenses, and a bridge. An interior portion of the lenses is covered with a mirror like reflective surface to reflect a user's eyes.

### FIELD OF THE INVENTION

The present invention relates generally to eyewear but more particularly to a relaxation and meditation eyewear.

### BACKGROUND OF THE INVENTION

In recent decades, meditation has become increasingly popular and many inventions have been created to facilitate that practice. One such device is a set of goggles or over sized glasses wherein the face where the lenses usually are is opaque and the inside of the goggles has flashing lights that a user can perceive even with eyes closed and which pulsate at different frequencies, presumably to match the frequency of the brain's alpha waves. These devices can be bulky and expensive. There has to be a cheaper alternative eyewear that can help achieve a meditative level.

### SUMMARY OF THE INVENTION

In view of the foregoing disadvantages inherent in the known devices now present in the prior art, the present invention, which will be described subsequently in greater detail, is to provide objects and advantages which are:

To provide for a low cost relaxation and meditation eyewear.

In order to do so, the invention comprises temples, opaque lenses, and a bridge. An interior portion of the lenses is covered with a mirror like reflective surface.

There is a crystal rock holder for holding a crystal, and an MP3 player holder.

An LED directed on the inside of the eyewear when ambient light is low.

The reflective surface consists in a removable insert.

The relaxation and meditation eyewear has a method of use consisting in the steps of an intended user putting on the eyewear while keeping his eyes open and looking into a reflection of his own eyes.

There has thus been outlined, rather broadly, the more important features of the invention in order that the detailed description thereof that follows may be better understood, and in order that the present contribution to the art may be better appreciated. There are additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

These together with other objects of the invention, along with the various features of novelty which characterize the invention, are pointed out with particularity in the claims annexed to and forming a part of this disclosure. For a better understanding of the invention, its operating advantages and the specific objects attained by its uses, reference should be made to the accompanying drawings and descriptive matter which contains illustrated preferred embodiment of the invention.

### BRIEF DESCRIPTION OF THE DRAWINGS

FIGS. 1A-B Isometric view of the invention with close up of nose bridge.

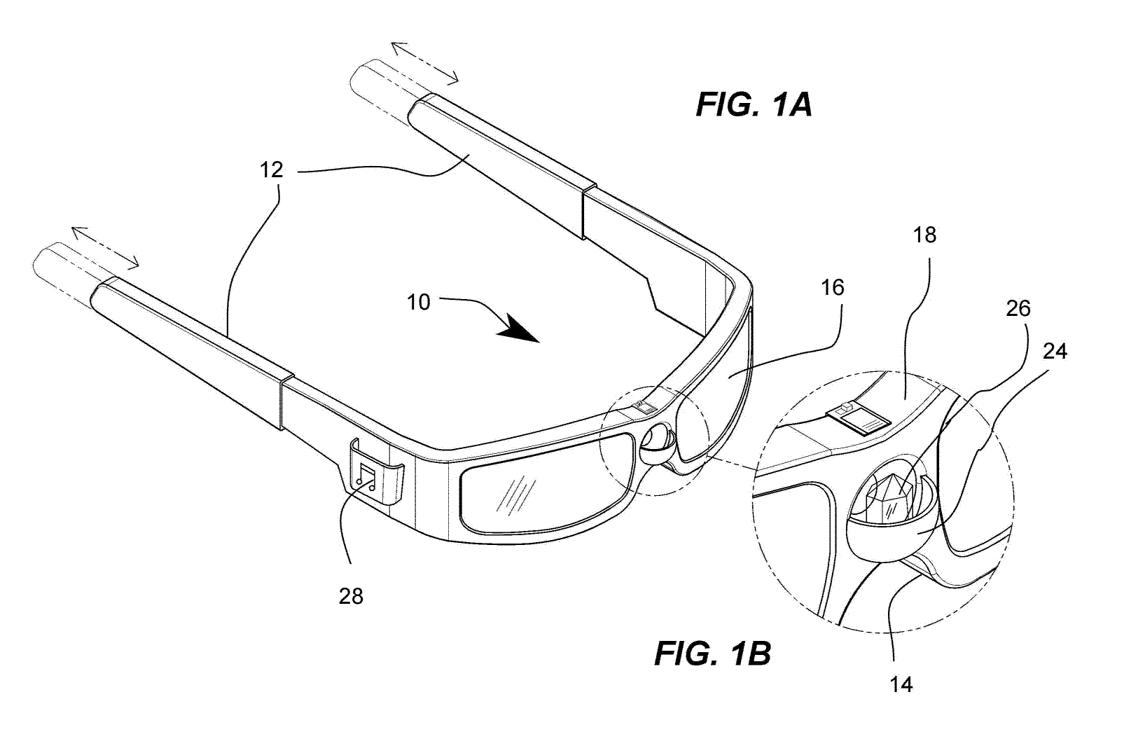

FIGS. 2A-B Reverse angle view featuring the interior of the invention, and close up of LED.

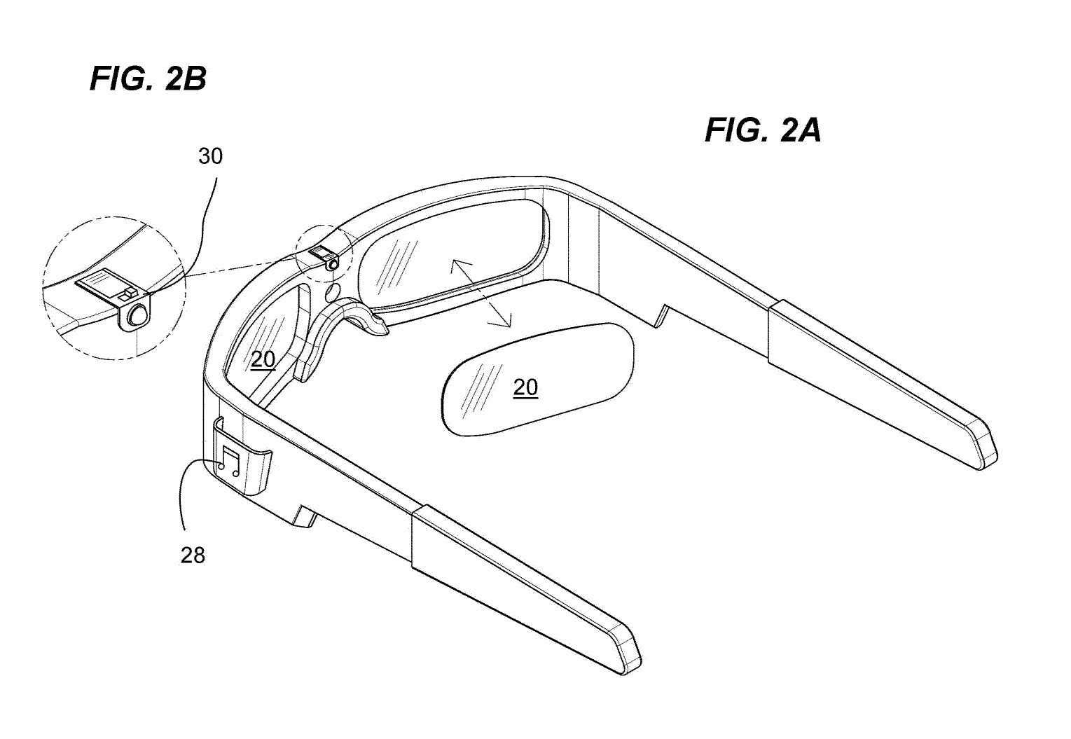

FIG. 3 Reverse angle view of a variant having changeable colored mirror inserts.

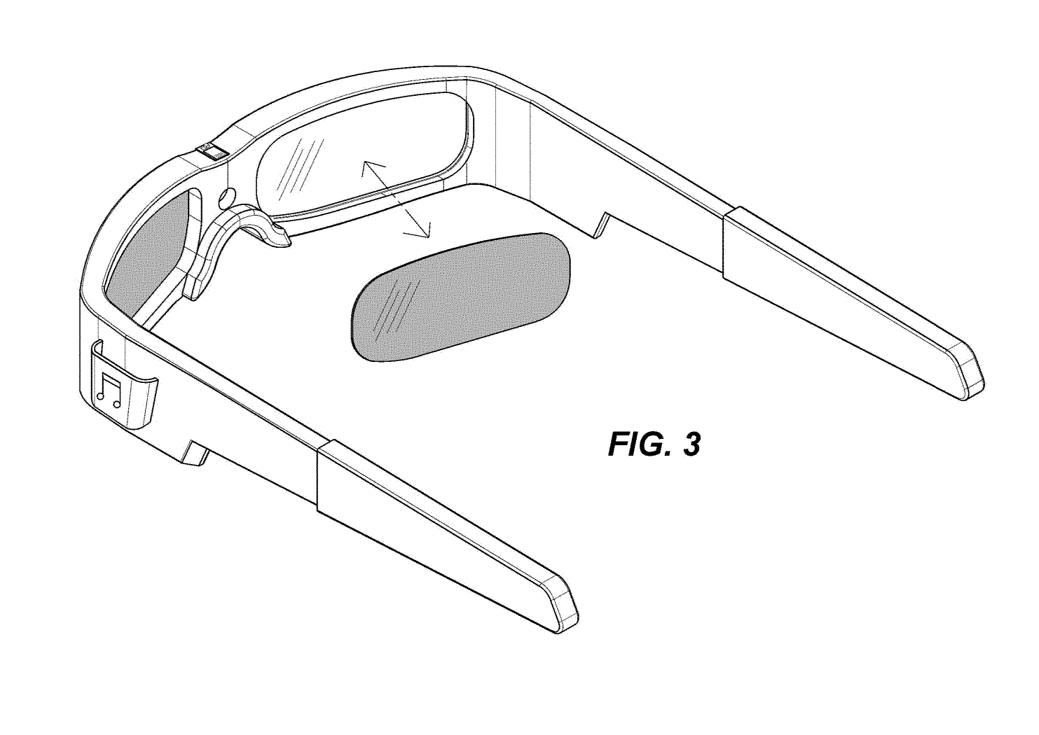

### DETAILED DESCRIPTION

A relaxation and meditation eyewear (10) comprises temples (12), opaque lenses (16), and a bridge (18). The inside of the lenses (16) are covered with a mirror like reflective surface (20).

Some of the features are used in existing eyewear such as adjustable temples (12) length, adjustable nose bridge (14).

Optionally, the eyewear (10) includes a crystal rock holder (24) for holding a crystal (26), an MP3 player holder (28). Also, an LED (30) directed on the inside adds some light so as to see one's reflection when in a dark place.

The eyewear (10) can be used as regular eyewear by removing the reflective surface (20) and putting in a translucent tinted or clear insert.

An intended user puts on the eyewear (10), keeps his eyes open and looks into the reflection of his own eyes.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention. Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1. A relaxation and meditation eyewear comprising temples, opaque lenses, and a bridge; an interior portion of said lenses being covered with a mirror like reflective surface.

2. The relaxation and meditation eyewear of claim 1 wherein there is a crystal rock holder for holding a crystal.

3. The relaxation and meditation eyewear of claim 1 wherein there is an MP3 player holder.

4. The relaxation and meditation eyewear of claim 1 wherein there is an LED directed on the inside of the eyewear.

5. The relaxation and meditation eyewear of claim 1 wherein said reflective surface consists in a removable insert.

6. The relaxation and meditation eyewear of claim 1 having a method of use consisting in the steps of an intended user putting on said eyewear while keeping his eyes open and looking into a reflection of his own eyes.
